(19) United States
(12) Patent Application Publication
(10) Pub. No.: US 2023/0218627 A1
(43) Pub. Date: Jul. 13, 2023

(54) PRESERVATIVE FREE PHARMACEUTICAL COMPOSITION FOR OPHTHALMIC ADMINISTRATION COMPRISING BRIMONIDINE

(71) Applicant: PHARMATHEN S.A., Pallini Attikis (GR)

(72) Inventors: Evangelos KARAVAS, Pallini Attikis (GR); Efthymios KOUTRIS, Pallini Attikis (GR); Vasiliki SAMARA, Pallini Attikis (GR); loanna KOUTRI, Pallini Attikis (GR); Anastasia KALASKANI, Pallini Attikis (GR); Andreas KAKOURIS, Pallini Attikis (GR); Anastasios KARATZAS, Pallini Attikis (GR); Manolis FOUSTERIS, Pallini Attikis (GR)

(21) Appl. No.: 17/923,330

(22) PCT Filed: May 5, 2021
(86) PCT No.: PCT/EP2021/025169

§ 371 Date: Nov. 4, 2022

(30) Foreign Application Priority Data
May 6, 2020 (GR) 20200100226

(51) Int. Cl. A61K 31/5377; A61K 31/498; A61K 9/08; A61K 47/32; A61K 47/02; A61K 9/00

(52) U.S. Cl. CPC A61K 31/5377; A61K 31/498; A61K 9/08; A61K 47/32; A61K 47/02; A61K 9/0048

(57) ABSTRACT

The present invention relates to a preservative free ophthalmic pharmaceutical formulation for topical administration containing a therapeutically effective quantity of Brimonidine or ophthalmological acceptable salts thereof alone or in combination with a therapeutically effective quantity of Timolol or ophthalmological acceptable salts thereof, to be used for the treatment of ocular hypertension and glaucoma.

### TECHNICAL FIELD OF THE INVENTION

The present invention relates to a preservative free ophthalmic formulation for topical administration containing a therapeutically effective quantity of Brimonidine or pharmaceutically acceptable salts thereof as a solely pharmaceutically active agent or in combination with Timolol or pharmaceutically acceptable salts thereof to be used for the treatment of elevated intraocular pressure (IOP) in patients with open angle glaucoma or ocular hypertension and process for the manufacturing thereof. Such preservative-free formulation is packed in container that ensures physical and chemical stability of the product.

### BACKGROUND OF THE INVENTION

Glaucoma is a group of eye disorders traditionally characterized by progressive damage to the eye, at least partly due to elevated intraocular pressure (IOP). It is the leading cause of irreversible blindness in the world and the second leading cause of vision loss after cataract, which is reversible surgically.

Eyes with glaucoma develop progressive peripheral visual field loss followed by central field loss. There are no anatomic factors that identify eyes that are at risk. Visual field loss cannot be recovered once it has occurred. Intraocular pressure (IOP) control remains the cornerstone in glaucoma management because modulating the IOP is the only proven strategy in reducing the risk of progressive retinal ganglion cell death. Ganglion cell death results in visual field loss and unacceptable quality of life.

Alpha-adrenergic receptors are divided into two types, alpha-1 and alpha-2, each having at least three subtypes. Although the precise activity of each subtype is not clearly understood, a number of specific ocular effects have been associated with the activation of the two different alpha-adrenergic receptor pathways. Ocular effects which are mediated by an alpha-2 adrenergic agonist include reduction of IOP and possibly neuroprotection of the optic nerve, while the corresponding effects for an alpha-1 adrenergic agonist include mydriasis, eyelid retraction and vasoconstriction.

Brimonidine is a highly selective alpha-2 adrenergic receptor agonist that is 1000-fold more selective for the alpha-2 adrenergic receptor than the alpha-1 adrenergic receptor and is classified as a third generation alpha-2 adrenergic receptor agonist with a higher selectivity than Clonidine or Apraclonidine, which are also alpha-2 adrenergic agonists. This characteristic gives the drug some therapeutic advantages, since it reduces the risk of systemic side effects, such as systemic hypotension, bradycardia, and sedation. In addition, there is a reduction in the risk for developing alpha-1 mediated ocular unwanted effects, such as conjunctival blanching, mydriasis, and eyelid retraction.

Brimonidine exerts its IOP-lowering effect via a dual mechanism: by inhibiting the enzyme adenylate cyclase, which reduces aqueous humor synthesis, while at the same time it moderately enhances outflow through the trabecular and the uveoscleral pathways.

Brimonidine Tartrate chemical name is 5-Bromo-N-(4,5-dihydro-1H-imidazol-2-yl)-6-quinoxalinamine tartrate and has molecular formula C₁₁H₁₀BrN₅·C₄H₆O₆. It is a pale yellow colored to wheatish colored powder with a molecular weight of 442.24 which is soluble in water and practically insoluble in anhydrous ethanol and in toluene.

Beta blockers such as Timolol are usually used as add-on therapy for patients who are already on Brimonidine therapy. Topical beta-blockers reduce the intraocular pressure (IOP) by blockade of sympathetic nerve endings in the ciliary epithelium causing a fall in aqueous humour production.

Timolol maleate is a white to almost white crystalline powder with a molecular weight of 432.5 and is soluble in water and ethanol. Its chemical name is (2S)-1-[(1,1-Dimethylethyl)amino]-3-[[4-(morpholin-4-yl)-1,2,5-thiadiazol-3-yl]oxy]propan-2-ol (Z)-butenedioate and has molecular formula C₁₇H₂₈N₄O₇S.

These two active ingredients decrease elevated IOP by complementary mechanisms of action and the combined effect results in additional IOP reduction compared to either compound administered alone. Thus, their combination has a rapid onset of action.

U.S. Pat. Nos. 4,195,085 and 4,861,760 describe the use of Timolol as an ophthalmic drug.

WO-A-2006/026215 discloses compositions useful for improving effectiveness of alpha-2-adrenergic agonist components including a nonionic solubility enhancing component.

Although attempts to provide stable solutions for ophthalmic administration have already been done, there still remains the need in the art for alternative formulations providing as well adequate chemical and physical characteristics and improved patient compliance. In particular, there is a need for formulations that are free from preservatives to be provided in a multiple use container and provide efficient dosing of the solution to the patient, without wastage.

### SUMMARY OF THE INVENTION

The present invention aims at developing aqueous pharmaceutical formulations for ophthalmic administration that overcome the disadvantages of and provide significant improvement over the prior art formulations.

More specifically, it is an object of the present invention to provide an aqueous eye drops solution comprising pharmacologically effective amount of Brimonidine or salts thereof and pharmaceutically acceptable excipients which is bioavailable and effective with sufficient self-life.

A further scope of the present invention is the preparation of an aqueous eye drops solution comprising combined pharmacologically effective amounts of Brimonidine or salts thereof and Timolol or salts thereof and pharmaceutically acceptable excipients resulting in a stable formulation matching the reference products both in vivo and in vitro.

It is, therefore, an object of the present invention to provide an efficient ophthalmic product that contains no antimicrobial preservatives. Such product is as effective in terms of therapy as products available with preservatives.

A further approach of the present invention is to provide ophthalmic solutions packed in container with appropriate design that are easily administrable in drop form. More specifically, the preservative free solution is packed in a multi dose preservative-free (MDPF) device.

Another aspect of the present invention is to provide a method for the preparation of a stable preservative free ophthalmic formulation containing a therapeutically effective quantity of Brimonidine or pharmaceutically acceptable salts thereof as a solely pharmaceutically active agent or in combination with Timolol or pharmaceutically acceptable salts thereof, permitting enhanced release of the active medicaments.

In accordance with the above objects of the present invention a method for the preparation of a preservative-free solution is provided comprising Brimonidine or salts thereof wherein both aseptic filtration and in-situ sterilization at 121° C. are applied to the preparation of finished drug product.

According to another embodiment of the present invention a method for the preparation of a preservative-free solution is provided comprising Brimonidine or salts thereof and Timolol or salts thereof wherein aseptic filtration is applied to the preparation of finished drug product.

Other objects and advantages of the present invention will become apparent to those skilled in the art in view of the following detailed description.

### DETAILED DESCRIPTION OF THE INVENTION

For the purposes of the present invention, a pharmaceutical composition comprising an active ingredient is considered to be “stable” if said ingredient degrades less or more slowly than it does on its own and/or in known pharmaceutical compositions.

Ocular administration of drugs is primarily associated with the need to treat ophthalmic diseases. Eye is the most easily accessible site for topical administration of a medication. Ophthalmic preparations are sterile products essentially free from foreign particles, suitably compounded and packaged for instillation into the eye. They are easily administered by the nurse or the patient himself, they have quick absorption and effect, less visual and systemic side effects, increased shelf life and better patient compliance.

Antimicrobial preservatives are added to aqueous preparations that are required to be sterile, such as in ophthalmic solutions. The use of preservatives in topical ophthalmic treatments is ubiquitous for any product that is to be used more than once by the patient as they prevent any microbes that may enter into the product after its first use from allowing those microbes to grow and infect the patient on a later use of the product. Although providing effective biocidal properties with well tolerated short-term use at low concentrations, preservatives can cause serious inflammatory effects on the eye with long-term use in chronic conditions, such as glaucoma or potentially ocular allergies.

Antimicrobial preservatives are not found in single use vials of ophthalmic solutions since they are manufactured aseptically or are sterilised and the products are used once and the dispenser is thrown away.

Preservative-free single dose containers most often are presented as blow-fill-seal (BFS) containers. The user takes the plastic vial and tears or cuts the plastic tip, inverts the vial and squeezes the ophthalmic liquid into the eye. Disadvantages of these systems are linked to the quite complicated filling technology, the need to overfill and amount of material needed for each dose. With an average drop size of ˜35 μl and the standard commercial volume of 400-500 μl, five times the required drug quantity ends up being discarded in case of single dose containers. Additionally, a big amount of packaging material is required associated with high manufacturing costs. A further disadvantage is that, despite numerous technical improvements were made by some manufacturers, the edges around the tip of the opened dropper of disposable, single-dose container are still very sharp, which may cause an accident to the patients eye.

As the use of preservative containing eye drops has been implicated in the development or worsening of ocular surface disease, there is a tendency to limit their use by reducing their concentration as much as possible in eye drops. The present invention provides completely preservative-free ophthalmic formulations. Such formulations are packed in containers that enable to deliver preservative-free formulations while providing shelf life similar to traditional formulations. The containers of the present invention ensure that medication is kept germ-free even after multiple uses.

Patient compliance is greatly increased as the pumps of the present invention permit them to use preservative-free eye drops without worrying about the potential side effects caused by some preservatives and the related short- and long-term consequences, such as pain or discomfort, foreign body sensation, stinging or burning, dry eye sensation, ocular surface breakdown.

We have found that the design of the tip of the container produce a highly accurate drop size with low variability of drop volume between each drop dispensed.

Therefore, we present as a feature of the present invention a multi-use ophthalmic product comprising a container with an integral bacterial protection system and which has a dispensing tip, wherein the ratio of the inner to the outer diameter of the dispensing tip is from 1:1 to 1:6, and the container having an ophthalmic composition that is dispensed from the tip into the eye of a patient wherein the ophthalmic composition is a preservative-free aqueous solution and contains pharmaceutically acceptable excipients.

Preservative-free solutions of the present invention are packed in a multi-dose preservative free (MDPF) container. The benefit for patients is that it can be used in similar way to “classic” squeezable multi-dose eye droppers. This device is able to calibrate droplets, which improves compliance.

To this bottle, two functions were implemented:
dispensing the drops while maintaining sterility
allowing air ingress inside the container to offset the volume dispensed while maintaining sterility of the content.

Preservatives are unnecessary if one can prevent the residual contents of the container from coming into contact with the outside environmental air not only after manufacture and during storage, but also during the period of application by the patient. This mean that all disadvantages associated with the use of preservatives particularly during long-term therapy would no longer apply if the container could preserve the solution sterile.

Tonicity refers to the osmotic pressure exerted by salts in aqueous solution. An ophthalmic solution is isotonic with another solution when the magnitudes of the colligative properties of the solutions are equal. An ophthalmic solution is considered isotonic when its tonicity is equal to that of 0.9% sodium chloride solution (290 mOsm). This requires that a certain tonicity agent be added so that the total osmotic pressure is the same as the body fluid. Sodium chloride, mannitol, dextrose, glycerine, potassium chloride are typical tonicity agents. Preferably, sodium chloride is used in the present invention as tonicity agent.

The present invention provides eye drops with ideal volume by controlling surface tension of the ophthalmic solution. The ophthalmic compositions of the present invention have surface tension of less than 70 mN/m and more than 50 mN/m at 25° C.

Preferred compositions are prepared using a buffering system that maintains the composition at a pH of about 6 to a pH of about 7, preferably 6.40-6.90.

Suitable buffering agents include, but are not limited to, dibasic sodium phosphate heptahydrate, citric acid monohydrate, monobasic sodium phosphate monohydrate, disodium phosphate dodecahydrate, hydrochloric acid, sodium hydroxide, sodium hydrogen carbonate, sodium citrate. Preferably, dibasic sodium phosphate heptahydrate, monobasic sodium phosphate monohydrate, citric acid monohydrate and sodium citrate are used in the present invention as buffering agents.

Viscosity is the property of resistance to flow in a fluid or semi fluid. It is an important parameter for ophthalmic compositions. A viscosity agent or thickening agent is a substance which can increase the viscosity of a liquid without substantially changing its other properties. Thickening agents are usually polymers of high molecular weight substance and the most commonly used are polyvinyl alcohol (PVA), hydroxy propyl methyl cellulose (HPMC), methyl cellulose.

The ophthalmic compositions of the present invention have viscosity of less than 20 cP at 25° C., more preferably the viscosity is less than 10 cP at 25° C. as measured by European Pharmacopoeia requirements (Capillary viscometer method; 01/2005:20209).

Poly(Vinyl Alcohol) (PVA) is a water-soluble synthetic polymer represented by the formula (C₂H₄₀)n. The n value for commercially available materials lies between 500 and 5000, equivalent to a molecular weight range of approximately 20 000-20 000. Its primary functional category for ophthalmic products is as viscosity-increasing agent for viscous formulations in a concentration range of 0.25-3.00%.

PVA is unique among the vinyl polymers in the fact that the monomer, vinyl alcohol, cannot be obtained in the quantities and purity required for polymerization purposes. As a result, it is manufactured by the polymerization of vinyl acetate and then converted by a hydrolysis process. Various grades of PVA are commercially available. The degree of polymerization and the degree of hydrolysis are the two determinants of their physical properties. Pharmaceutical grades are partially hydrolyzed materials, with the unconverted fractions being poly(vinyl Acetate). and are named according to a coding system. For example, each grade of EMD Millipore's PVAs has two groups of numbers separated by a dash. The first number represents the dynamic viscosity (in mPa·s or cP) of a 4% w/v aqueous solution at 20° C. as a relative indication of the molar mass. The second number is the degree of hydrolysis of poly(vinyl Acetate). Poly(vinyl alcohols) used in the present invention include PVA 4-88, PVA 18-88, PVA 40-88.

Preferably poly(vinyl alcohol) grade 40-88 is used as viscosity agent in the present invention in an amount of 1.4% w/v.

### EXAMPLES

The formulation development of Brimonidine tartrate PF solution started with a composition comprising the following excipients: citric acid monohydrate; sodium citrate dihydrate; sodium chloride; poly(vinyl alcohol); pH adjusting agents (NaOH and/or HCl) and water for injections.

The manufacturing process followed is described below:

### Preparation of Solution A

1. In a clean vessel of appropriate size, an adequate quantity of water for injection, corresponding to the 60% of total batch size, is added:
2. The appropriate amounts of sodium chloride, citric acid monohydrate and sodium citrate dihydrate are added to the vessel and completely dissolved under stirring:
3. The appropriate amount of Brimonidine Tartrate is added to the vessel and completely dissolved under stirring:
4. The solution pH is adjusted to 6.40 with sodium hydroxide and/or hydrochloric acid aqueous solutions of appropriate normalities;
5. The resulting solution is filtered through a sterilizing grade filter (PVDF 0.2 μm) under aseptic conditions.

### Preparation of Solution B

6. In a separate clean vessel, another quantity of water for injection, corresponding to ⅓ (approx. 33%) of total batch size, is added:
7. The appropriate amount of PVA is added to the vessel and initially is stirred for 20 min at RT;
8. The above mixture is heated up to 80-90° C. and stirred for 30 min to ensure homogeneity:
9. The resulting solution is in-situ sterilized in a double jacketed stainless steel tank at 121° C. for 30 min.

### Preparation of Final Solution

10. The above solutions A and B are quantitatively mixed under aseptic conditions and stirred to ensure homogeneity;
11. If necessary, the solution pH is adjusted to 6.40 with sodium hydroxide and/or hydrochloric acid aqueous solutions of appropriate normalities:
12. The final solution is adjusted with the addition of water for injections and the solution pH is checked.

As already mentioned a variety of PVA grades exist that differentiate at chain molecular weights (as a consequence at the viscosity) and at the hydrolysis degree. Hydrolysis grade mainly relates to mechanical properties (e.g. mechanical strength and adhesiveness), while a low hydrolysis grade may facilitate the incorporation of higher amounts of a poorly water-soluble API. However, Brimonidine Tartrate is soluble in water at given concentrations and PVA is incorporated in final formulation as a viscosity agent. Thus, a high hydrolysis grade PVA (88%) was chosen with limited water insoluble poly(vinyl acetate) content in order any PVA solubility concern to be minimized.

Three different PVA grades were chosen according to their viscosity values of a 4% w/v aqueous solution (40 g/L), as listed in Table 1 below.

TABLE 1
PVA grades and nominal viscosity values

| | Nominal viscosity* |
|---|---|
| PVA Grade | [4% w/v (40 g/L)] |
| PVA 4-88 | 3.4-4.6 cP |
| PVA 18-88 | 15.3-20.7 cP |
| PVA 40-88 | 34.0-46.0 cP |

*Viscosity specifications set as per supplier's CoAs

The above excipients were studied in Trials 1-3 described below in Table 2. According to FDA Inactive Ingredient Database, the highest formula percentage for PVA (ophthalmic route—solution/drops dosage form) is 1.4% w/v and this concentration was set for initial trials. The composition of other components as well as manufacturing process remained constant as described above.

TABLE 2
Quantitative formulae of formulation Trials 1-3

| | Trial 1 | Trial 2 | Trial 3 |
|---|---|---|---|
| Component | % w/v | | |
| Brimonidine Tartrate | 0.200 | 0.200 | 0.200 |
| [Equivalent to Brimonidine] | 0.130 | 0.130 | 0.130 |
| PVA 4-88 | 1.40 | — | — |
| PVA 18-88 | — | 1.40 | — |
| PVA 40-88 | — | — | 1.40 |
| Citric acid monohydrate | 0.110 | 0.110 | 0.110 |
| Sodium citrate dihydrate | 0.450 | 0.450 | 0.450 |
| Sodium chloride | 0.600 | 0.600 | 0.600 |

| | |
|---|---|
| NaOH/HCl aqueous solutions | q.s. to pH 6.40 |
| Water for injections | q.s. to 100 |

The physicochemical analysis of the above trials is presented below in Table 3.

TABLE 3
Physicochemical properties of Trials 1-3

| Test | Trial 1 | Trial 2 | Trial 3 |
|---|---|---|---|
| pH | 6.4 | 6.4 | 6.4 |
| Specific gravity | 1.011 | 1.012 | 1.012 |
| Surface tension (mN/m) | 54.9 | 54.9 | 53.4 |
| Osmolality (mOsm/kg) | 270 | 269 | 272 |
| Buffering capacity (mmol/L)/ΔpH | 3.5 | 3.7 | 3.9 |
| Viscosity (cP) [100 rpm-spindle 00] | 1.6 | 2.7 | 3.9 |

Considering that different PVA grades were applied among Trials 1-3, various viscosity values were recorded for each finished formulation trial PVA acts as a viscosity agent and its contribution in all other physicochemical properties is negligible. Regarding viscosity values, the PVA 40-88 grade was selected for further trials since the viscosity value of Trial 3 formulation is satisfactory.

After the determination of PVA grade, three more formulations were prepared in order to adjust the buffer capacity. Trials 1-3 have low buffer capacity values and the concentrations of the two buffering agents have to be optimized so an increased buffer capacity to be achieved. For this reason the total citrate concentration was increased by adding more citric acid monohydrate, as shown in

Table 4 below. All other components remain at constant concentrations.

TABLE 4
Quantitative formulae of formulation Trials 4-6

| | Trial 4 | Trial 5 | Trial 6 |
|---|---|---|---|
| Component | % w/v | | |
| Brimonidine Tartrate | 0.200 | 0.200 | 0.200 |
| [Equivalent to Brimonidine] | 0.130 | 0.130 | 0.130 |
| PVA 40-88 | 1.40 | 1.40 | 1.40 |
| Citric acid monohydrate | 0.120 | 0.125 | 0.130 |
| Sodium citrate dihydrate | 0.450 | 0.450 | 0.450 |
| Sodium chloride | 0.600 | 0.600 | 0.600 |

| | |
|---|---|
| NaOH/HCl aqueous solutions | q.s. to pH 6.40 |
| Water for injections | q.s. to 100 |

The physicochemical properties of Trials 4-6 are shown in Table.

TABLE 5
Physicochemical properties of Trials 4-6

| Test | Trial 4 | Trial 5 | Trial 6 |
|---|---|---|---|
| pH | 6.4 | 6.4 | 6.4 |
| Specific gravity | 1.012 | 1.011 | 1.011 |
| Surface tension (mN/m) | 53.6 | 53.1 | 52.8 |
| Osmolality (mOsm/kg) | 275 | 278 | 280 |
| Buffering capacity (mmol/L)/ΔpH | 3.5 | 8.7 | 10.9 |
| Viscosity (cP) [100 rpm-spindle 00] | 3.9 | 3.9 | 3.9 |

According to the results, all buffer capacities are acceptable, however Trial 5 is the optimum formulation trial and is selected for the next development trials.

The final quality attribute to be optimized is osmolality. Sodium chloride concentration was investigated by three more trials in order the targeted osmolality value to be achieved. The compositions of Trials 7-9 are shown below. Only the Sodium chloride concentration ranges, all other component concentrations remain constant.

TABLE 6
Quantitative formulae of formulation Trials 7-9

| | Trial 7 | Trial 8 | Trial 9 |
|---|---|---|---|
| Component | % w/v | | |
| Brimonidine Tartrate | 0.200 | 0.200 | 0.200 |
| [Equivalent to Brimonidine] | 0.130 | 0.130 | 0.130 |
| PVA 40-88 | 1.40 | 1.40 | 1.40 |
| Citric acid monohydrate | 0.125 | 0.125 | 0.125 |
| Sodium citrate dihydrate | 0.450 | 0.450 | 0.450 |
| Sodium chloride | 0.620 | 0.650 | 0.680 |

| | |
|---|---|
| NaOH/HCl aqueous solutions | q.s. to pH 6.40 |
| Water for injections | q.s. to 100 |

The physicochemical properties of Trials 7-9 are shown in Table 7 below.

TABLE 7
Physicochemical properties of Trials 7-9

| Test | Trial 7 | Trial 8 | Trial 9 |
|---|---|---|---|
| pH | 6.4 | 6.4 | 6.4 |
| Specific gravity | 1.012 | 1.012 | 1.013 |
| Surface tension (mN/m) | 53.1 | 52.5 | 54.5 |
| Osmolality (mOsm/kg) | 285 | 298 | 310 |
| Buffering capacity (mmol/L)/ΔpH | 9.0 | 8.5 | 8.8 |
| Viscosity (cP) [100 rpm-spindle 00] | 3.9 | 4.0 | 3.9 |

Concerning the results, Trial 8 exhibits satisfactory physicochemical characteristics and its composition is procured for further study of drug product manufacturing process as well as stability testing.

Within the formulation development the manufacturing process applied comprises the preparation of a sterile drug product by using two different sterilization methods. More particularly, the PVA solution (Solution B) was sterilized through in-situ sterilization at 121° C. for 30 min, while the solution including API & inactive ingredients (Solution A) was sterilized by aseptic filtration through a sterilizing grade filter. After the sterilization of aforementioned solutions all the other steps were conducted through aseptic processing conditions. The final solution is filled under aseptic conditions and the appropriate multi-dose preservative free container (Novelia® packaging system) is sealed.

With regards to PVA solution (4.2% w/v aqueous solution), the impact of in-situ sterilization on viscosity value of solution was evaluated prior & after sterilization. More particularly, as shown in

Table 8, the viscosity of Solution B was recorded before & after in-situ sterilization and it can be stated that the risk of sterilization method on viscosity value is considered low. Taking into account that the viscosity of finished product is determined by this one of intermediate product (PVA solution-Solution B), it is obvious that the impact of current sterilization method on viscosity of PF finished product can also be considered low.

TABLE 8
In situ sterilization process impact on viscosity values

| | Viscosity | Viscosity |
|---|---|---|
| | Before in-situ | After in-situ |
| | sterilization | sterilization |
| PVA 40-88 solution | 38.5 cP | 37.7 cP |
| (4.2% w/v) | | |
| Brimonidine 0.2% | 4.1 cP | 4.0 cP |
| PF formulation Trial | | |

Except physicochemical properties, which have been monitored during formulation development, assay & related substances of finished product are also evaluated. The zero time results are presented in Table 9 below.

TABLE 9
Zero time results

| | BRIMONIDINE TARTRATE 2 mg/mL |
|---|---|
| Test Parameters | Preservative Free eye drops, solution |
| Appearance | Clear, greenish-yellow to light greenish- |
| | yellow solution |
| pH | 6.4 |
| Osmolality (mOsm/kg) | 294 |
| Viscosity (cP) | 3.9 |
| Surface tension (mN/m) | 55.0 |
| Buffering capacity | 9.8 |
| (mmol/L)/ΔpH | |
| Specific gravity | 1.012 |
| Assay Brimonidine | 99.7% |
| (95.0%-105.0%) | |

Related substances of Brimonidine Tartrate

| | |
|---|---|
| Any known Impurity (NMT | ND |
| 1.0%) | |
| Impurity G (NMT 3.0%) | ND |
| Any unknown Impurity | ND |
| (NMT 1.0%) | |
| Total impurities (NMT 6.5%) | 0.09% |

The formulation trial of Brimonidine Tartrate 2 mg/mL Preservative Free eye drops solution was packed in MDPF containers (Novelia® packaging system) and the stability profile was monitored under different storage conditions (long-term & accelerated conditions) described in the following Tables.

TABLE 10
Stability study (25° C. ± 2° C./RH: 60% ± 5%)
Brimonidine Tartrate 2 mg/mL Preservative-Free, Eye drops solution

| | | Time points (months) | | |
|---|---|---|---|---|
| | | (25° C. ± 2° C./RH: 60% ± 5%) | | |
| Control Tests | Specifications | 0 | 3 | 6 |
| pH | 5.9-6.9 | 6.4 | 6.5 | 6.4 |
| Osmolality | 300 mOsm/Kg ± | 294 | 299 | 293 |
| | 10% (270-330 | | | |
| | mOsm/Kg) | | | |
| Assay of | 95.0-105.0% of | 99.7% | 100.2% | 99.8% |
| Brimonidine | the stated amount | | | |
| Tartrate | of Brimonidine | | | |
| Related | Any known | Known | Known | Known |
| Substances | Impurity: | Impurity: | Impurity: | Impurity: |
| and | NMT1.0% | ND | ND | ND |
| degradation | Impurity | Impurity | Impurity | Impurity |
| products of | G: NMT3.0% | G: ND | G: 0.07% | G: 0.14% |
| Brimonidine | Any unknown | Total | Max. | Max. |
| tartrate | Impurity: | 0.09% | Unknown: | Unknown: |
| | NMT1.0% | | 0.08% | 0.06% |
| | Total | | Total | Total |
| | Impurities: | | 0.15% | 0.32% |
| | NMT6.5% | | | |
| Sterility | Sterile | Complies | Complies | Complies |

TABLE 11
Stability study (40° C. ± 2° C./RH: 75% ± 5%)
Brimonidine Tartrate 2mg/mL Preservative-Free, Eye drops solution

| | | Time points (months) | | |
|---|---|---|---|---|
| | | (40° C. ± 2° C./RH: 75% ± 5%) | | |
| Control Tests | Specifications | 0 | 3 | 6 |
| pH | 5.9-6.9 | 6.4 | 6.4 | 6.4 |
| Osmolality | 300 mOsm/Kg ± | 294 | 298 | 296 |
| | 10% (270-330 | | | |
| | mOsm/Kg) | | | |
| Assay of | 95.0-105.0% of | 99.7% | 100.0% | 98.6% |
| Brimonidine | the stated amount | | | |
| Tartrate | of Brimonidine | | | |
| Related | Any known | Known | Known | Known |
| Substances | Impurity: | Impurity: | Impurity: | Impurity: |
| and | NMT1.0% | ND | ND | ND |
| degradation | Impurity | Impurity | Impurity | Impurity |
| products of | G: NMT3.0% | G: ND | G: 0.25% | G: 0.57% |
| Brimonidine | Any unknown | Total | Max. | Max. |
| tartrate | Impurity: | 0.09% | Unknown: | Unknown: |
| | NMT1.0% | | 0.07% | 0.09% |
| | Total | | Total | Total |
| | Impurities: | | 0.34% | 0.72% |
| | NMT6.5% | | | |
| Sterility | Sterile | Complies | Complies | Complies |

Considering the results of stability study, Brimonidine Tartrate 2 mg/mL Preservative Free eye drops, solution drug product displays acceptable quality attributes under both storage conditions studied. Both assay and impurity profile, even under accelerated conditions (40° C.±2° C./RH: 75%±5%) were well within specifications. Thus, it can be stated that current manufacturing process & packaging system are considered suitable for Brimonidine Tartrate 2 mg/mL PF drug product.

Two more procedures were evaluated. Both manufacturing processes also include the preparation of solutions A and B, but differ in sterilization methods applied.

Along the second manufacturing process the final ophthalmic solution is sterilized through aseptic filtration through a sterilizing grade filter (0.2 μm). API solution (Solution A) can be successfully filtered through the sterilizing grade filter without any absorption and/or adsorption phenomena leading to a final solution of acceptable assay & impurities profile. However, the viscous nature of PVA solution leads to filter clogging. Even though different filter membranes were evaluated (i.e. Hydrophilic PVDF, PES etc.), Solution B seems to exhibits incompatibility with filter membrane and consequently, aseptic filtration was rejected as a potential sterilization method for finished product. Thus, such manufacturing process is not recommended for the manufacturing of Brimonidine Tartrate 2 mg/mL PF drug product.

The third manufacturing process consisted of moist heat sterilization and aseptic processing of final bulk solution. Since terminal sterilization is not feasible for the filled vials of finished product (due to deformation issues of LDPE-based packaging components), the moist heat sterilization method (121° C. for 15 min) was selected along the current manufacturing process for the sterilization of bulk ophthalmic solution.

The high temperature level of moist heat sterilization may affect the drug product quality attributes and thus it has to be thoroughly evaluated. The zero time results are listed in Table 12 below.

TABLE 12
Zero time results

| | BRIMONIDINE TARTRATE |
|---|---|
| | 2 mg/mL Preservative Free |
| Test Parameters | eye drops, solution |
| Appearance | Clear, greenish-yellow to light greenish- |
| | yellow solution |
| pH | 6.4 |
| Osmolality (mOsm/kg) | 285 |
| Viscosity (cP) | 4.0 |
| Surface tension (mN/m) | 56.3 |
| Buffering capacity | 7.1 |
| (mmol/L)/ΔpH | |
| Specific gravity | 1.012 |
| Assay Brimonidine | 99.8% |
| (95.0%-105.0%) | |

Related substances of Brimonidine Tartrate

| | |
|---|---|
| Any known Impurity (NMT | ND |
| 1.0%) | |
| Impurity G (NMT 3.0%) | 2.3% |
| Any unknown Impurity | Max. |
| (NMT 1.0%) | Unknown: 1.1% |
| Total impurities (NMT 6.5%) | 3.3% |

Although all physicochemical properties after moist heat sterilization are within the acceptance rages, an unknown impurity is recorded at an out of specification level, while known Impurity G percentage was increased by 2.3% (compared to formulation trial prepared under first manufacturing process, Table 9) As a consequence, total impurities percentage has been raised to 3.3% indicating the impact of moist heat cycle at the quality of finished product.

Considering the above, the moist heat sterilization is not recommended for the manufacturing of Brimonidine Tartrate 2 mg/mL PF drug product.

Overall, the first manufacturing process, consisting of in-situ sterilization of PVA solution and aseptic filtration of Brimonidine Tartrate API solution is considered suitable for the manufacturing of preservative-free ophthalmic product.

The preferred preservative-free ophthalmic composition comprising Brimonidine according to the present invention is illustrated in Table 13 below:

TABLE 13
Brimonidine Tartrate PF composition

| Component | Concentration (% w/v) |
|---|---|
| Brimonidine Tartrate | 0.200 |
| [Equivalent to Brimonidine] | 0.130 |
| PVA 40-88 | 1.400 |
| Citric acid monohydrate | 0.125 |
| Sodium citrate dihydrate | 0.450 |
| Sodium chloride | 0.650 |
| NaOH/HCl aqueous solutions | q.s. to pH 6.40 |
| Water for injections | q.s. to 100.000 |

The physicochemical properties of Brimonidine Tartrate 0.2% w/v PF preferred composition of the present invention are summarized below.

TABLE 14
Physicochemical properties of Brimonidine Tartrate PF

| | Brimonidine Tartrate PF |
|---|---|
| Test | Eye drops, solution |
| pH | 6.4 |
| Specific gravity | 1.012 |
| Surface tension (mN/m) | 55.0 |
| Osmolality (mOsm/kg) | 298 |
| Buffering capacity | 8.5 |
| (mmol/L)/ΔpH | |
| Viscosity (cP) | 4.0 |
| [100rpm-spindle 00] | |
| Average Delivery | 34.2 |
| Volume (μL/drop) | |

The formulation development of Brimonidine tartrate/Timolol maleate combination PF solution started with a composition comprising the following excipients: sodium phosphate dibasic heptahydrate, sodium phosphate monobasic dihydrate; sodium hydroxide, hydrochloric acid and water for injections.

Considering the above, the compositions of both phosphate salts were optimized by a 2² full factorial DoE study. The responses studied were buffer capacity (Y1) and osmolality (Y2) values. The composition levels (% w/v) of the two phosphate salts were set at three different levels (High, Medium, Low) The 2² full factorial DoE study is summarized in Table 15 below.

TABLE 15
DoE study for phosphate salts level

| | | Levels | | |
|---|---|---|---|---|
| Factors: Phosphate salts | | −1 | 0 | +1 |
| A | Na2HPO4•7H2O (% w/v) | 2.35 | 2.45 | 2.55 |
| B | NaH2PO4•2H20 (% w/v) | 0.10 | 0.25 | 0.40 |

| | | |
|---|---|---|
| Responses | Goal | Acceptable ranges |

| | | | |
|---|---|---|---|
| Y1 | Buffering capacity | Acceptable | 40.0-55.0 |
| | (mmol/L)/ΔpH | | |
| Y2 | Osmolality | Acceptable | 280-295 |
| | (mOsm/kg) | | |

The manufacturing process followed is described below:

Solution Preparation
1. In a clean vessel of appropriate size, an adequate quantity of water for injection, corresponding to the 90% of total batch size, is added:
2. The appropriate amounts of sodium phosphate dibasic heptahydrate (Na₂HPO₄·O₇H₂O) and sodium phosphate monobasic dihydrate (NaH₂PO₄·2H₂O) are added to the vessel and completely dissolved under stirring;
3. The appropriate amount of Timolol Maleate is added to the vessel and completely dissolved under stirring:
4. The appropriate amount of Brimonidine Tartrate is added to the vessel and completely dissolved under stirring;
5. If necessary, the solution pH is adjusted to 6.90 with sodium hydroxide and/or hydrochloric acid aqueous solutions of appropriate normalities,
6. The final solution is adjusted with the addition of water for injections and the solution pH is checked.
7. The final solution is filtered through a sterilizing grade filter (PVDF 0.2 μm) under aseptic conditions.
8. The final solution is filled under aseptic conditions and the appropriate multi-dose preservative free container (Novelia® packaging system) is sealed.

The physicochemical analysis, regarding the two studied responses, for all trials are given in Table 16 below.

TABLE 16
Experimental results of DoE study

| | | | | Responses | |
|---|---|---|---|---|---|
| | | Factors | | Buffering | |
| | | Na₂HPO₄•7H₂O | NaH₂PO₄•2H₂0 | capacity | Osmolality |
| Run Order | Pattern | (% w/v) | (% w/v) | (mmol/L)/ΔpH | (mOsm/kg) |
| 1 | −− | 2.35 | 0.100 | 42.2 | 268 |
| 2 | ++ | 2.55 | 0.400 | 52.2 | 322 |
| 3 | −+ | 2.35 | 0.400 | 49.4 | 304 |
| 4 | +− | 2.55 | 0.100 | 42.2 | 286 |
| 5 | 00 | 2.45 | 0.250 | 47.3 | 295 |
| 6 | 00 | 2.45 | 0.250 | 46.9 | 291 |
| 7 | −+ | 2.35 | 0.400 | 48.9 | 300 |
| 8 | +− | 2.55 | 0.100 | 42.0 | 284 |
| 9 | ++ | 2.55 | 0.400 | 51.6 | 319 |
| 10 | −− | 2.35 | 0.100 | 41.8 | 265 |

Since center points were included in DoE study, the significance of the curvature effect was tested using an adjusted model. The ANOVA results for Buffer capacity and Osmolality are presented in Table 17 and Table, respectively.

TABLE 17
Analysis of Variance for Buffer capacity vs Phosphate salts

| | | Sum of | Mean | F | P | |
|---|---|---|---|---|---|---|
| Source | df* | Squares | Square | Value | value | Comments |
| Model | 4 | 152.75 | 38.19 | 362.66 | <0.05 | Significant |
| Linear | 2 | 148.18 | 74.09 | 703.63 | <0.05 | |
| Na2HPO4•7H2O | 1 | 4.28 | 4.28 | 40.62 | <0.05 | |
| (% w/v) | | | | | | |
| NaH2PO4•2H20 | 1 | 143.91 | 143.91 | 1366.63 | <0.05 | |
| (% w/v) | | | | | | |
| 2-way | 1 | 3.58 | 3.58 | 33.98 | <0.05 | |
| interactions | | | | | | |
| Curvature effect | 1 | 0.99 | 0.99 | 9.39 | <0.05 | |
| Pure Error | 5 | 0.53 | 0.11 | | | |
| Total | 9 | 153.28 | | | | |

| | |
|---|---|
| R-sq | 99.7% |
| R-sq (adj) | 99.4% |

*df: degrees of freedom

TABLE 18
Analysis of Variance for Osmolality vs Phosphate salts

| | | Sum of | Mean | F | P | |
|---|---|---|---|---|---|---|
| Source | df* | Squares | Square | Value | value | Comments |
| Model | 4 | 3205.40 | 801.35 | 148.40 | <0.05 | Significant |
| Linear | 2 | 3205.40 | 1602.50 | 296.76 | <0.05 | |
| Na₂HPO₄•7H₂O | 1 | 684.50 | 684.50 | 126.76 | <0.05 | |
| (% w/v) | | | | | | |
| NaH₂PO₄•2H₂0 | 1 | 2520.50 | 2520.50 | 466.76 | <0.05 | |
| (% w/v) | | | | | | |
| 2-way | 1 | 0.00 | 0.00 | 0.00 | 1.000 | No |
| interactions | | | | | | Significant |
| Curvature effect | 1 | 0.40 | 0.40 | 0.07 | 0.796 | |
| Pure Error | 5 | 27.00 | 5.40 | | | |
| Total | 9 | 3232.40 | | | | |

| | |
|---|---|
| R-sq | 99.2% |
| R-sq (adj) | 98.5% |

*df: degrees of freedom

Based on the results, both phosphate salts affect significantly the two physicochemical responses. However, the impact of sodium phosphate monobasic dihydrate (NaH₂PO₄·2H₂O) is higher on both quality properties. Medium level for dibasic sodium phosphate. Na₂HPO₄·7H₂O (Factor A), which corresponds to 2.45% w/v concentration, and a concentration close to high level of monobasic sodium phosphate (Factor B). i.e. 0.360% w/v, are considered suitable for current formula to achieve satisfactory osmolality & buffering capacity.

Two manufacturing procedures were evaluated. In the first one the final solution is filtered through a sterilizing grade filter (PVDF 0.2 μm) under aseptic conditions while in the second one the final solution is sterilized through a moist heat cycle at 121° C. for 15 min. In both cases the final solution is filled under aseptic conditions and the appropriate multi-dose preservative free container (Novelia® packaging system) is sealed.

Aseptic filtration was applied during formulation development of current invention. Except physicochemical properties, which have been monitored during the formulation development trials, assay and related substances were also evaluated. The zero time results are presented in Table 19 below.

TABLE 19
Zero time results for Aseptic filtered preparation

| | BRIMONIDINE TARTRATE/TIMOLOL |
|---|---|
| | 0.2%/0.5% w/v |
| Test Parameters | Preservative Free eye drops, solution |
| Appearance | Clear, greenish-yellow solution |
| pH | 6.9 |
| Osmolality (mOsm/kg) | 296 |
| Viscosity (cP) | 1.4 |
| Surface tension (mN/m) | 65.3 |
| Buffering capacity | 50.0 |
| (mmol/L)/ΔpH | |
| Specific gravity | 1.017 |
| Assay Brimonidine | 98.9% |
| (95.0%-105.0%) | |
| Assay Timolol | 98.7% |
| (95.0%-105.0%) | |

Related substances of Brimonidine Tartrate

| | |
|---|---|
| Any known | ND |
| impurity (NMT 1.0%) | |
| Impurity G (NMT 3.0%) | ND |
| Any unknown | 0.10% |
| impurity (NMT 1.0%) | |
| Total impurities | 0.10% |
| (NMT 6.5%) | |

Related substances of Timolol

| | |
|---|---|
| Any known | 0.01% |
| impurity (NMT 1.0%) | |
| Total impurities | 0.01% |
| (NMT 3.0%) | |

Enantiomeric Purity of Timolol

| | |
|---|---|
| Impurity A (NMT 1.0%) | ND |

The formulation trial of Brimonidine Tartrate-Timolol 2 mg/mL+5 mg/mL Preservative Free eye drops solution product was packed in MDPF containers (Novelia® packaging system) and the stability profile was monitored under different storage conditions (long-term & accelerated conditions) described in the following Tables.

TABLE 20
Stability study (25° C. ± 2° C./RH: 60% ± 5%)
Brimonidine Tartrate-Timolol 2 mg/mL-5 mg/mL
Preservative-Free, Eye drops solution

| | | Time points (months) | | |
|---|---|---|---|---|
| | | (25° C. ± 2° C./RH: 60% ± 5%) | | |
| Control Tests | Specifications | 0 | 3 | 6 |
| pH | 6.5-7.3 | 6.9 | 7.0 | 6.9 |
| Osmolality | 290 mOsm/Kg ± | 296 | 294 | 297 |
| | 10% (260-320 | | | |
| | mOsm/Kg) | | | |
| Assay of | 95.0-105.0% of | 98.9% | 97.6% | 98.5% |
| Brimonidine | the stated | | | |
| Tartrate | amount of | | | |
| | Brimonidine | | | |
| Assay of | 95.0-105.0% of | 98.7% | 97.7% | 99.6% |
| Timolol | the stated | | | |
| | amount of | | | |
| | Timolol | | | |
| Related | Any known | Known | Known | Known |
| Substances | Impurity: | Impurity: | Impurity: | Impurity: |
| and | NMT1.0% | ND | ND | ND |
| degradation | Impurity | Impurity | Impurity | Impurity |
| products of | G: NMT3.0% | G: 0.07% | G: 0.22% | G: 0.30% |
| Brimonidine | Any unknown | Total | Max. | Max. |
| tartrate | impurity: | 0.26% | Unknown: | Unknown: |
| | NMT1.0% | | 0.46% | 0.36% |
| | Total | | Total | Total |
| | Impurities: | | 0.90% | 0.95% |
| | NMT6.5% | | | |
| Related | Any known | Known | Known | Known |
| Substances | Impurity: | Impurity: | Impurity: | Impurity: |
| and | NMT1.0% | 0.01% | 0.01% | 0.01% |
| degradation | Any unknown | Max. | Max. | Max. |
| products of | Impurity: | Unknown: | Unknown: | Unknown: |
| Timolol | NMT1.0% | 0.02% | 0.03% | 0.08% |
| Maleate | Total | Total | Total | Total |
| | impurities: | 0.04% | 0.05% | 0.11% |
| | NMT3.0% | | | |
| Enantiomeric | Impurity | Impurity | Impurity | Impurity |
| Purity | A: NMT 1.0% | A: ND | A: 0.09% | A: 0.12% |
| of Timolol | | | | |
| Sterility | Sterile | Complies | Complies | Complies |

TABLE 21
Stability study (40° C. ± 2° C./RH: 75% ± 5%)
Brimonidine Tartrate-Timolol 2 mg/mL-5 mg/mL
Preservative-Free, Eye drops solution

| | | Time points (months) | | |
|---|---|---|---|---|
| | | (40° C. ± 2° C./RH. 75% ± 5%) | | |
| Control Tests | Specifications | 0 | 3 | 6 |
| pH | 6.5-7.3 | 6.9 | 6.8 | 6.9 |
| Osmolality | 290 mOsm/Kg ± | 298 | 297 | 294 |
| | 10% (260-320 | | | |
| | mOsm/Kg) | | | |
| Assay of | 95.0-105.0% of | 98.9% | 97.9% | 96.2% |
| Brimonidine | the stated | | | |
| Tartrate | amount of | | | |
| | Brimonidine | | | |
| Assay of | 95.0-105.0% of | 98.7% | 98.6% | 97.9% |
| Timolol | the stated | | | |
| | amount of | | | |
| | Timolol | | | |
| Related | Any known | Known | Known | Known |
| Substances | Impurity: | Impurity: | Impurity: | Impurity: |
| and | NMT1.0% | ND | ND | ND |
| degradation | Impurity | Impurity | Impurity | Impurity |
| products of | G: NMT3.0% | G: 0.07% | G: 0.68% | G: 1.9% |
| Brimonidine | Any unknown | Total | Max. | Max. |
| tartrate | Impurity: | 0.26% | Unknown: | Unknown: |
| | NMT1.0% | | 0.51% | 0.59% |
| | Total | | Total | Total |
| | Impurities: | | 1.8% | 4.2% |
| | NMT6.5% | | | |
| Related | Any known | Known | Known | Known |
| Substances | Impurity: | Impurity: | Impurity: | Impurity: |
| and | NMT1.0% | 0.01% | 0.03% | 0.03% |
| degradation | Any unknown | Max. | Max. | Max. |
| products of | Impurity: | Unknown: | Unknown: | Unknown: |
| Timolol | NMT1.0% | 0.02% | 0.15% | 0.19% |
| Maleate | Total | Total | Total | Total |
| | Impurities: | 0.04% | 0.24% | 0.25% |
| | NMT3.0% | | | |
| Enantiomeric | Impurity A: | Impurity | Impurity | Impurity |
| Purity | NMT 1.0% | A: ND | A: 0.14% | A: 0.17% |
| of Timolol | | | | |
| Sterility | Sterile | Complies | Complies | Complies |

Considering the results of stability study. Brimonidine Tartrate—Timolol 2 mg/mL-5 mg/mL preservative free eye drops, solution product packed in MDPF containers and manufactured through aseptic filtration displays acceptable quality attributes under all stability conditions. Both APIs assay and impurity profiles were well within specification ranges even under accelerated conditions (40° C.±2° C./RH: 75%±5%). Thus, aseptic filtration through a sterilizing grade filter (PVDF 0.2 μm) is considered suitable for the manufacturing of Brimonidine Tartrate-Timolol PF drug product.

Moist Heat Sterilization is the next process to be assessed. The zero time results for moist-heat sterilized preparation are listed in Table 22 below.

TABLE 22
Zero time results for moist heat-sterilized preparation

| | BRIMONIDINE TARTRATE/TIMOLOL |
|---|---|
| | 0.2%/0.5% w/v |
| Test Parameters | Preservative Free eye drops, solution |
| Appearance | Clear, greenish-yellow solution |
| pH | 6.9 |
| Osmolality (mOsm/kg) | 303 |
| Viscosity (cP) | 1.4 |
| Surface tension (mN/m) | 65.1 |
| Buffering capacity | 50.0 |
| (mmol/L)/ΔpH | |
| Specific gravity | 1.017 |
| Assay Brimonidine | 102.3% |
| (95.0%-105.0%) | |
| Assay Timolol | 99.0% |
| (95.0%-105.0%) | |

Related substances of Brimonidine Tartrate

| | |
|---|---|
| Any known | ND |
| Impurity (NMT 1.0%) | |
| Impurity G (NMT 3.0%) | 3.2% |
| Unknown Impurities | Max. Unknown |
| (NMT 1.0%) | RRT0.65: 1.1% |
| Total impurities | 5.6% |
| (NMT 6.5%) | |

Related substances of Timolol

| | |
|---|---|
| Any known | 0.37% |
| Impurity (NMT 1.0%) | |
| Total impurities | 0.37% |
| (NMT 3.0%) | |

Enantiomeric Purity of Timolol

| | |
|---|---|
| Impurity A (NMT 1.0%) | 0.29% |

As per above results, all physicochemical properties are well within the acceptance ranges and similar to the aseptic filtered trial. However, the impurity levels are increased, especially these ones related to Brimonidine Tartrate. Both known impurity G and unknown impurities were found to be out of specification.

Considering the current results, the moist heat sterilization is not recommended for the manufacturing of Brimonidine Tartrate-Timolol 2 mg/mL+5 mg/mL PF drug product. As a result, aseptic filtration is considered suitable for the manufacturing of preservative-free ophthalmic product since both zero time and stability data are well within acceptance ranges. The preferred preservative-free ophthalmic composition comprising Brimonidine-Timolol according to the present invention is illustrated in Table 23 below:

TABLE 23
Brimonidine Tartrate-Timolol Maleate PF composition

| Component | Composition (% w/v) |
|---|---|
| Brimonidine Tartrate | 0.200 |
| [Equivalent to Brimonidine] | 0.130 |
| Timolol Maleate | 0.683 |
| [Equivalent to Timolol] | 0.500 |
| Sodium phosphate, dibasic | 2.450 |
| heptahydrate | |
| (Na2HPO4•7H2O) | |
| Sodium phosphate, monobasic | 0.360 |
| dihydrate | |
| (NaH2PO4•2H2O) | |
| NaOH/HCl aqueous solutions | q.s. to pH 6.90 |
| Water for injections | q.s. to 100.000 |

The physicochemical properties of Brimonidine Tartrate-Timolol Maleate 0.2%-0.5% PF preferred composition of the present invention are summarized below (Table 24).

TABLE 24
Physicochemical properties of Brimonidine
Tartrate-Timolol Maleate PF

| | Brimonidine Tartrate-Timolol PF |
|---|---|
| Test | Eye drops, solution |
| pH | 6.9 |
| Specific gravity | 1.016 |
| Surface tension | 65.2 |
| (mN/m) | |
| Osmolality | 294 |
| (mOsm/kg) | |
| Buffering capacity | 50.0 |
| (mmol/L)/ΔpH | |
| Viscosity (cP) | 1.4 |
| [100rpm-spindle 00] | |
| Average Delivery | 30.9 |
| Volume (μL/drop) | |

While the present invention has been described with respect to the particular embodiments, it will be apparent to those skilled in the art that various changes and modifications may be made in the invention without departing from the spirit and scope thereof, as defined in the appended claims.

1. A preservative-free ophthalmic pharmaceutical composition comprising Brimonidine or pharmaceutically acceptable salts thereof as a solely pharmaceutically active agent or in combination with Timolol or pharmaceutically acceptable salts thereof.

2. The preservative free ophthalmic pharmaceutical composition according to claim 1, comprising Brimonidine tartrate in a quantity of 0.2% w/v.

3. The preservative free ophthalmic pharmaceutical composition according to claim 1, comprising Timolol maleate in a quantity of 0.683% w/v.

4. The pharmaceutical composition according to claim 1 further comprising adequate quantity of pharmaceutically acceptable excipients selected so as to provide the following physical parameters to the solution:
a) viscosity of less than 10 cP at 25° C. as measured by European Pharmacopoeia requirements (Capillary viscometer method; 01/2005:20209);
b) surface tension of less than 70 mN/m and more than 50 mN/m at 25° C.

5. The pharmaceutical composition according to claim 1 comprising Brimonidine Tartrate and a viscosity agent in an amount of maximum 1.4% w/v.

6. The pharmaceutical composition according to claim 5, wherein the viscosity agent is poly(vinyl alcohol) grade 40-88.

7. The pharmaceutical composition according to claim 6, wherein it further comprises citric acid monohydrate, sodium citrate dihydrate, sodium chloride, hydrochloric acid, sodium hydroxide and water of injections.

8. The pharmaceutical composition according to claim 7, wherein the pH value is about 6.4.

9. The pharmaceutical composition according to claim 1, comprising Brimonidine Tartrate, Timolol Maleate, Sodium phosphate dibasic heptahydrate, Sodium phosphate monobasic dihydrate, hydrochloric acid, sodium hydroxide and water of injections.

10. The pharmaceutical composition according to claim 9, wherein the pH value is about 6.9.

11. The pharmaceutical composition of claim 1 wherein the composition is an eye drops solution packed in a MDPF container.

12. A process for preparing a preservative-free pharmaceutical composition for ophthalmic administration comprising Brimonidine Tartrate is provided comprising the following steps:
Preparation of Solution A
In a clean vessel of appropriate size, an adequate quantity of water for injection, corresponding to the 60% of total batch size, is added;
The appropriate amounts of sodium chloride, citric acid monohydrate and sodium citrate dihydrate are added to the vessel and completely dissolved under stirring;
The appropriate amount of Brimonidine Tartrate is added to the vessel and completely dissolved under stirring;
The solution pH is adjusted to 6.40 with sodium Hydroxide and/or hydrochloric acid aqueous solutions of appropriate normalities;
The resulting solution is filtered through a sterilizing grade filter (PVDF 0.2 μm) under aseptic conditions.
Preparation of Solution B
In a separate clean vessel, another quantity of water for injection, corresponding to ⅓ (approx. 33%) of total batch size, is added;
The appropriate amount of PVA is added to the vessel and initially is stirred for 20 min at RT;
The above mixture is heated up to 80-90° C. and stirred for 30 min to ensure homogeneity;
The resulting solution is in-situ sterilized in a double jacketed stainless steel tank at 121° C. for 30 min.
Preparation of final Solution
The above solutions A and B are quantitatively mixed under aseptic conditions and stirred to ensure homogeneity;
If necessary, the solution pH is adjusted to 6.40 with sodium Hydroxide and/or hydrochloric acid aqueous solutions of appropriate normalities;
The final solution is adjusted with the addition of water for injections and the solution pH is checked.
The final solution is filled under aseptic conditions and the appropriate multi-dose preservative free container is sealed.

13. The manufacturing process of preparation of claim 12 wherein both aseptic filtration and in-situ sterilization at 121° C. are applied to the preparation of finished drug product.

14. A process for preparing a preservative-free pharmaceutical composition for ophthalmic administration comprising Brimonidine Tartrate in combination with Timolol Maleate is provided comprising the following steps:
In a clean vessel of appropriate size, an adequate quantity of water for injection, corresponding to the 90% of total batch size, is added;
The appropriate amounts of sodium phosphate dibasic heptahydrate (Na2HPO4·7H2O) and sodium phosphate monobasic dihydrate (NaH2PO4·2H2O) are added to the vessel and completely dissolved under stirring;
The appropriate amount of Timolol Maleate is added to the vessel and completely dissolved under stirring;
The appropriate amount of Brimonidine Tartrate is added to the vessel and completely dissolved under stirring;
If necessary, the solution pH is adjusted to 6.90 with sodium hydroxide and/or hydrochloric acid aqueous solutions of appropriate normalities;
The final solution is adjusted with the addition of water for injections and the solution pH is checked;
The final solution is filtered through a sterilizing grade filter (PVDF 0.2 μm) under aseptic conditions.
The final solution is filled under aseptic conditions and the appropriate multi-dose preservative free container is sealed.

15. The manufacturing process of preparation of claim 14 wherein aseptic filtration is applied to the preparation of finished drug product.
